(19) United States
(12) Patent Application Publication
(10) Pub. No.: US 2023/0233484 A1
(43) Pub. Date: Jul. 27, 2023

(54) USE OF VITAMIN K IN PREVENTING OR COUNTERACTING COVID-19 DISEASE AND DIAGNOSTIC TEST TO ESTIMATE THE RISK OF DEVELOPING SEVERE DISEASE OR MORTALITY BY COVID-19

(71) Applicant: EMPHYSEMA SOLUTIONS BV, Beuningen (NL)

(72) Inventor: Rob Janssen, Beuningen (NL)

(73) Assignee: EMPHYSEMA SOLUTIONS BV, Beuningen (NL)

(21) Appl. No.: 17/917,835

(22) PCT Filed: Apr. 9, 2021
(86) PCT No.: PCT/NL2021/050236

§ 371 Date: Oct. 7, 2022

(30) Foreign Application Priority Data
Apr. 9, 2020 (NL) 2025326
Apr. 25, 2020 (NL) 2025427
May 29, 2020 (NL) 2025715

(51) Int. Cl. A61K 31/122; A61K 31/593; A61P 31/14

(52) U.S. Cl. CPC A61K 31/122; A61K 31/593; A61P 31/14

(57) ABSTRACT

A composition is provided comprising a therapeutically active amount of vitamin K for administering to a subject as prophylactic for preventing or reducing the risk of developing severe disease or mortality by COVID-19 or a similar infectious disease, or as therapeutic for preventing said disease becoming more severe or reducing the severity of said disease. Also provided is a diagnostic test to estimate the risk of developing severe disease or mortality by COVID-19 or a similar infectious disease in a subject involving assessing vitamin K status in blood, serum or plasma of said subject.

### FIELD OF THE INVENTION

The present invention is in the fields of diagnostics, nutrition and pharmacotherapy. In particular, the invention relates to a new use of vitamin K in pharmaceutical or nutraceutical compositions for preventing or counteracting Covid-19 disease and/or alleviating severe symptoms of said disease. The invention relates also to a diagnostic test to estimate the risk of developing severe disease or mortality by Covid-19 in a subject involving assessing vitamin K status in blood, serum or plasma of said patient.

### BACKGROUND OF THE INVENTION

Coronavirus disease 2019 (Covid-19) is an infectious disease caused by severe acute respiratory syndrome (SARS) coronavirus (CoV)-2.¹ The majority of individuals who contract SARS-CoV-2 have mild symptoms.² However, a significant proportion develops respiratory failure due to pneumonia and/or acute respiratory distress syndrome (ARDS).³ Covid-19 may also have extrapulmonary manifestations. Coagulopathy and venous thromboembolism are prevalent in severe SARS-CoV-2 infections and are associated with decreased survival.^{4,5} The mechanisms that activate coagulation in Covid-19 are not known at present but appear to be linked to inflammatory responses rather than specific properties of the virus.

The 2019-20 coronavirus pandemic is an ongoing pandemic of Covid-19. The outbreak started in Wuhan, Hubei province, China, as early as November 2019. The World Health Organization (WHO) declared the outbreak to be a Public Health Emergency of International Concern on 30 Jan. 2020 and recognized it as a pandemic on 11 Mar. 2020. As of 29 May 2020, approximately 5.94 million cases of Covid-19 have been reported in 215 countries and territories, resulting in approximately 362,700 deaths.

The virus is mainly spread during close contact and by small droplets produced when those infected are coughing, sneezing or talking. These droplets may also be produced during breathing. Coronavirus is most contagious during the first three days after onset of symptoms, although spread may be possible before symptoms appear and in later stages of the disease.

Common symptoms include fever, cough and shortness of breath. The most significant manifestations of COVID-19 include pulmonary and coagulopathic complications. The former may lead to respiratory failure and death. The latter may lead to thrombosis and embolism. The time from exposure to onset of symptoms is typically around five days but may range from two to 14 days.

Both pulmonary and thrombotic manifestations of COVID-19 have been linked to hyperinflammation. Pro-inflammatory cytokines - and particularly IL-6 - have been consistently associated with more severe disease.

The pandemic has led to severe global socioeconomic disruption, the postponement or cancellation of sporting, religious, political and cultural events, and widespread shortages of supplies exacerbated by panic buying. Schools and universities have closed either on a nationwide or local basis in 193 countries, affecting approximately 99.4 percent of the world’s student population.

Primary treatment is symptomatic and supportive therapy. However, the severe global socioeconomic and medical crisis will only end if an effective vaccine becomes available that prevents all genetic variants of Covid-19, or if a treatment becomes available that prevents the development of severe disease and mortality in SARS-CoV-2-infected individuals.

Coagulation is an intricate balance between clot promoting and dissolving processes in which vitamin K plays a well-known role. Vitamin K may occur in two different main forms: K1 and K2. Whereas K1 comprises one single chemical structure (phylloquinone), K2 is a group name for the family of menaquinones (abbreviated as MK-n), which have in common a methylated naphthoquinone ring structure as the functional group, but which vary in the length of their polyisoprenoid side chain. In the generally adopted nomenclature, n stands for the number of isoprenyl residues in MK-n. The number of isoprenyl residues in the side chain may vary from 1 (in MK-1) to 13 (in MK-13). The different forms of vitamin K share the function as coenzyme for the posttranslational enzyme gammaglutamate carboxylase (GCCX), but substantial differences have been reported with respect to absorption, transport, and pharmacokinetics {Schurgers L J, Vermeer C. Biochim Biophys Acta 1570 (2002) 27-32}. Whereas K1 is preferentially utilized by the liver, K2 vitamins (mainly the long-chain menaquinones MK-7 through MK-10) are readily transported to extra-hepatic tissues, such as bone, arteries, lungs and adipose tissue. Commercially available K-vitamins include K1, MK-4 and MK-7.

Coagulation factors II (FII; i.e. prothrombin), VII, IX and X depend on vitamin K for carboxylation to fulfil their primary biological function. Vitamin K is also cofactor of anticoagulant proteins C and S. In contrast to vitamin K-dependent procoagulant factors and protein C, a significant proportion of protein S is extrahepatically synthesized in endothelial cells, which plays a local suppressive role against thrombosis formation in blood vessels.⁶ Carboxylation during vitamin K deficiency is more severely compromised for extrahepatic than hepatic vitamin K-dependent proteins (FIG. 1).⁷ This can paradoxically lead to enhanced thrombogenicity in a state of low vitamin K.⁸

The product of vitamin K action is the unusual amino acid gammacarboxy-glutamic acid, abbreviated as Gla. Presently, 17 Gla-containing proteins have been discovered and in those cases in which their functions are known they play key roles in regulating important physiological processes, including haemostasis, calcium metabolism, and cell growth and survival {Berkner K L, Runge K W. J Thromb Haemostas 2 (2004) 2118-2132}. Since new Gla-proteins are discovered almost every second year {Viegas C S et al. Am J Pathol 175 (2009) 2288-2298}, it is to be expected that more Gla-protein-controlled processes will be identified in the near future. In all Gla-proteins the function of which is known, the Gla-residues are essential for the activity and functionality of these proteins, whereas proteins lacking these residues are defective {Berkner K L, Runge K W. J Thromb Haemostas 2 (2004) 2118-2132}. The specificity with which Gla-domain structures facilitate interaction of vitamin K-dependent coagulation proteins with cell membranes is now becoming understood {Huang M et al. Nature Struct Biol 10 (2003) 751-756}. Likewise, it is well accepted that the Gla-residues of osteocalcin confer binding of the protein to the hydroxyapatite matrix of bone in a manner strongly suggestive of selectivity and functionality {Hoang Q Q. Nature 425 (2003) 977-980}.

The Gla-proteins involved in haemostasis are all synthesized in the liver: four blood coagulation factors (II, VII, IX, and X) and three coagulation inhibiting proteins (C, S, and Z). In the normal healthy population, vitamin K intake is sufficient to cover the requirements of the liver, so in healthy adults all coagulation factors are fully carboxylated.

Matrix Gla protein (MGP) is also vitamin K-dependent but not involved in coagulation.⁹ MGP is well-known as a calcification inhibitor in arterial walls.¹⁰ However, it is also strongly expressed in lungs.¹¹ MGP’s role in the pulmonary compartment seems to be comparable with that in the vasculature.¹² Elastic fibers are essential components of the extracellular matrix in lungs and have high affinity for calcium.^{13,14} MGP is crucial for the protection of elastic fibers against calcification.⁹ Degradation, fibrosis and mineralization of elastic fibers are interrelated remodeling processes, as synthesis of matrix metalloproteinases (MMPs) and release of latent transforming growth factor (TGF)-β from the extracellular matrix are enhanced in parallel with elastic fiber calcification.¹⁵⁻¹⁷These processes also involve partially degraded elastic fibers becoming prone to mineralization due to increased polarity.¹⁴

Both carboxylated (cMGP) and uncarboxylated MGP (ucMGP) species have been detected in normal human plasma {Cranenburg E C et al. Thromb. Haemostas. 104 (2010) 811-822; Schurgers L J et al. Blood 109 (2007) 3279-3283}. It was found that notably the fraction known as desphospho-uncarboxylated MGP (dp-ucMGP) can be used as a marker for vascular vitamin K status and as a “risk marker” directly associated with future artery calcification risk and cardiovascular mortality {Cranenburg E C et al. Thromb. Haemostas. 101 (2009) 359-366; Schurgers L J et al. Clin. J. Am. Soc. Nephrol. 5 (2010) 568-575}.

Besides for dp-ucMGP also conformation-specific assays for OC are commercially available [carboxylated OC (cOC) and uncarboxylated OC (ucOC)], respectively), and are often used to link bone vitamin K status with osteoporotic bone loss and fracture risk. The evidence that poor vitamin K status is associated with accelerated bone loss, low bone mass and osteoporosis is overwhelming and generally accepted {Szulc P J et al. J. Clin. Invest. 91 (1993) 1769-1774; Luukinen H et al. J. Bone Miner. Res. 15 (2000) 2473-2478}. Also, serum ucOC is broadly used as a surrogate marker for overall vitamin K status.

Conformation-specific assays for MGP have been described by Vermeer C, inter alia in the patent literature (European Patent Nos. 1190259 and 1591791, U.S. Pat. Nos. 6,746,847, 7,700,296 and 8,003,075). Their diagnostic use for cardiovascular disease and rheumatoid arthritis has been demonstrated.

Dietary vitamin K intake has been associated with inflammation. High vitamin K1 intake and high circulating vitamin K1 levels were found to be correlated with low levels of the inflammation marker CRP {Shea M K et al. Am J Epidemiol 167 (2008) 313-320}. The authors indicate that the mechanism may not be based on the classical function of vitamin K (posttranslational protein carboxylation) but on increased expression of inflammation-related genes.

It is known that most extra-hepatic Gla-proteins are substantially under-carboxylated with 20-30% of the total antigen being present in the Gla-deficient (and hence inactive) state. Examples are the bone Gla-protein osteocalcin (OC) and the vascular Matrix Gla-Protein (MGP) {Knapen M H et al. Ann Int Med 111 (1989) 1001-1005; Cranenburg EC et al. Thromb Haemostas 104 (2010) 811-822}. Whereas the function of MGP as an inhibitor of soft tissue calcification (e.g. in arteries and lungs) is well understood {Schurgers L J et al. Thromb Haem 100 (2008) 593-603}, the function of OC has remained a matter of debate even 30 years after its discovery, but most data presently available indicate that it has a function in the deposition of hydroxyapatite crystals in the inorganic bone matrix. The main function of the more recently discovered Gla-rich protein (GRP) is probably also related to inhibiting tissue calcification, notably in cartilage {Cancella M L et al. Adv Nutr 3 (2012) 174-181}. Recent findings suggest that GRP action may not remain restricted to cartilage, however.

Although multiple proposals in the pharmaceutical field have been made to contain the Covid-19 pandemic and to reduce the risk for preventing disease severity and patient mortality, none of them have shown sufficient efficacy to completely prevent severe disease. Therefore, there is still a need for improvement, since morbidity and mortality remain unacceptable high in the art of COVID-19. The present invention provides such an improvement.

### BRIEF DESCRIPTION OF THE DRAWINGS

FIG. 1 is a schematic drawing (not part of the invention) representing the distribution of vitamin K1 in the body. (1) After absorption, vitamin K1 is preferentially transported to the liver via the portal circulation, where it is utilized for carboxylation of hepatic coagulation factors. This implies that during periods of vitamin K insufficiency, (2) the grade of carboxylation is usually higher for hepatic factor II and other procoagulant factors (3) than for endothelial protein S in veins and pulmonary matrix Gla protein (MGP).

FIG. 2 is a schematic drawing representing the assumed sequential pathologic steps linking SARS-CoV-2 pneumonia to vitamin K insufficiency and accelerated elastic fiber degradation.(1) Severe acute respiratory syndrome coronavirus-2 (SARS-CoV-2) enters alveolar type II (AT2) cell. (2) The infected AT2 cell responses by upregulating synthesis of proinflammatory cytokines such as IL-6. (3) This leads to an increase in the number and activation of pulmonary macrophages. (4) These infiltrating macrophages produce matrix metalloproteinases (MMPs) (5), which leads to accelerated degradation of elastic fibers (5a) and thereby the release of desmosine from these fibers (5b) leading to elevated desmosine levels in lungs and blood. (6) The increased polarity of partially degraded elastic fibers (7) enhances their affinity for calcium, and consequently, leads to increased elastic fiber calcium content. (7a) MMP synthesis is upregulated in parallel with calcium content, which further accelerates elastic fiber degradation in a self-propagating vicious circle. (8) Matrix Gla protein (MGP) synthesis is upregulated in an attempt to protect elastic fibers from calcification and degradation, (8a) which means that need for vitamin K to activate additional MGP increases. (8b) This increased utilization of vitamin K may induce vitamin K insufficiency, (9) in which case increased production of MGP in a state of vitamin K insufficiency leads to increased desphospho-uncarboxylated (dp-uc)MGP in lungs and blood.

FIGS. 3A and 3B are plots showing circulating dp-ucMGP and PIVKA-II in Covid-19 patients. (3A) Dp-ucMGP was measured in plasma of Covid-19 patients with a good outcome (discharge without mechanical ventilation, n=74, orange) or poor outcome (mechanical ventilation and/or death, n=60, red), compared to a cohort of healthy controls. Subjects with high dp-ucMGP have low extrahepatic vitamin K status and vice versa. The maximal dp-ucMGP measured during the study is shown, with open circles representing those patients using VKA at admission. (3B) PIVKA-II was measured in plasma at baseline in those patients not using VKA (n=121). The normal range for healthy controls is shown in gray.

FIGS. 4A and 4B are plots showing the correlation between dp-ucMGP and desmosine. (4A) For all Covid-19 patients who were not dialysis-dependent at admission with a good outcome (discharge without mechanical ventilation, n=73, orange) or poor outcome (mechanical ventilation and/or death, n=58, red) log-transformed baseline dp-ucMGP and desmosine values are shown, with open circles representing VKA users. The black line represents a linear regression analysis. (4B) Scatterplot showing circulating desmosine levels in those patients over 40 years old (n=128) by age, the black line represents the deduced equation for Covid-19 patients. The green and blue lines represent Huang et al’s calculated equations for non-smoking and smoking controls, respectively.

FIG. 5 is a plot showing IL-6 levels in hospitalized patients with Covid-19. IL-6 levels were measured in plasma from 133 patients. Patients outcome was defined as “good” when they survived without the need of invasive ventilation, or “bad” when they needed supportive invasive ventilation and/or deceased. There is a significant difference between the two groups (p<0.0001).

FIGS. 6A and 6B are plots showing the effects of vitamin K status on IL-6. (6A) shows IL-6 levels compared with different levels of dp-ucMGP. (6B) shows the correlation between IL-6 and dp-ucMGP, both log transformed. Dp-ucMGP levels measured in patients with COVID-19 were divided into three groups: 1) dp-ucMGP plasma levels 0-1000 pmol/L, 2) dp-ucMGP plasma levels 1000-2000 pmol/L and 3) dp-ucMGP plasma levels >2000 pmol/L. IL-6 levels were compared with those three groups of dp-ucMGP levels. There was a significant difference between the first group of dp-ucMGP (0-1000 pmol/L) and the second and third group (1000-2000 pmol/L and >2000 pmol/L), p=0.0004. There is no significant difference between the second and third group. There was a significant correlation between dp-ucMGP and IL-6 (Pearson r=0.121, p=0.0001). Besides this there is a significant relationship between dp-ucMGP and IL-6 in the linear regression model.

FIGS. 6C and 6D are plots showing the effects of vitamin 25(OH) D status on IL-6. (6C) shows IL-6 levels compared with different levels of vitamin 25(OH) D. (6D) shows the correlation between IL-6 and vitamin 25(OH) D, both log transformed. The line shows the result of linear regression. Vitamin D levels were compared with IL-6 levels in the same way as dp-ucMGP and was also divided into three groups: 1) 0-25 nmol/L, 2) 25-50 nmol/L and 3) >50 nmol/L. There was no significant difference between the three groups of vitamin D levels and IL-6 (Kruskal-Wallis p=0.4774). There is a significant correlation between vitamin D and IL-6 (Pearson r=0.021, p=0.0499) but there is no significant relationship found in the linear regression model (p=0.0999).

FIG. 7 is a plot showing the effect on elastic fiber degradation. Correlation between IL-6 and desmosine, both log transformed. The line shows the resukt of a linear regression. To look at the correlation between IL-6 and elastic fiber degradation, Pearson’s test was used. There is a significant correlation between IL-6 and desmosine (p=0.0017).

### DEFINITIONS

The term “vitamin K”, as used herein, refers to phylloquinone (also known as vitamin K₁); and menaquinone (also known as vitamin K₂). Within the group of vitamin K2, special reference is made to menaquinone-4 (MK-4) and the long-chain menaquinones (MK-7, MK-8 and MK-9), in particular menaquinone-7 (MK-7). It is generally accepted that the naphthoquinone moiety which they have in common is the functional group, so that the mechanism of action is similar for all K vitamins. Differences may be expected, however, with respect to intestinal absorption, transport, tissue distribution, and bioavailability.

As used herein, the terms “effective amount” and “therapeutically effective amount” are interchangeable and refer to an amount that results in bringing the plasma concentration of uncarboxylated Gla-proteins within the normal range, preferably around the lower-normal value.

As used herein, the term “vitamin K status” refers to the extent to which various Gla-proteins have been carboxylated. Poor vitamin K status means that the dietary vitamin K intake is insufficient to ensure complete Gla-protein carboxylation. Both ucOC and dp-ucMGP are well recognized as sensitive markers for poor vitamin K status.

In general a distinction is made between hepatic vitamin K status (carboxylation of coagulation factors) and extra-hepatic vitamin K status (carboxylation of Gla-proteins not synthesized in the liver). The liver produces the vitamin K-dependent blood coagulation factors. Insufficient hepatic vitamin K status is extremely rare; therefore, the clotting factors are no sensitive markers for vitamin K status.

MGP originates from tissues other than from the liver, mainly from arteries and cartilage. Likewise, OC originates primarily from bone. In the present patent application a person’s vitamin K status will be regarded as poor when the circulating levels of uncarboxylated extra-hepatic Gla-proteins MGP (measured as dp-ucMGP) and/or osteocalcin (as measured as ucOC) are is above the upper normal level in healthy adults. Evidence is provided that there is a strong correlation between the circulating levels of ucOC and dp-ucMGP, demonstrating that both species are markers for poor vitamin K status and suggesting that other extra-hepatic Gla-proteins may be similarly used for the same purpose.

As used herein, the term “study cohort” is defined as the population (group of subjects, group of patients) in which the particular study has been performed.

For the purposes of this specification and the appended claims, unless otherwise indicated, all numbers expressing quantities of ingredients, percentages or proportions of materials, reaction conditions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters set forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a monomer” includes two or more monomers.

### SUMMARY OF THE INVENTION

In one aspect of the present invention a composition is provided for use in preventing or counteracting Covid-19 disease or a similar disease and/or alleviating severe symptoms of said disease, said composition comprising a therapeutically active amount of vitamin K, either alone or in combination with one or more other therapeutically active agents, wherein the use comprises administering said composition to a mammalian subject, either as prophylactic agent in preventing or reducing the risk of developing a serious disease or mortality by COVID-19 or similar microbial infectious disease in the said subject, or as therapeutic agent in preventing that the said disease becomes more severe, or as therapeutic agent in reducing the severity of the said disease.

In a further aspect of the invention certain preferred embodiments of said composition are defined and claimed in dependent claims 2 to 14.

In another aspect of the present invention a diagnostic test is provided for estimating the risk of developing severe disease or mortality by COVID-19 or a similar infectious disease in a subject involving assessing vitamin K status in blood, serum or plasma of said subject as defined in claim 15.

In still another aspect of the invention certain preferred embodiments of said diagnostic test are defined and claimed in dependent claims 16 to 18.

These and other aspects of the present invention will be more fully outlined in the detailed description which follows.

### DETAILED DESCRIPTION OF THE INVENTION

The present invention is based on the surprising finding that vitamin K deficiency is associated with Covid-19 related morbidity and mortality, in particular in patients with comorbidities including diabetes, cardiovascular diseases and renal disease.

We found that extrahepatic vitamin K status was severely reduced in Covid-19 patients, as reflected by elevated inactive MGP, and related to poor outcome. Procoagulant prothrombin activation by vitamin K remained preserved in the majority of Covid-19 patients. Impaired MGP activation was linked to accelerated elastic fiber degradation and premorbid vascular calcifications. About fifty percent of total anticoagulant protein S is activated extra-hepatically by vitamin K, and this activation is likely also impaired during Covid-19. Given the fact that procoagulant activity remains intact, this is compatible with the increased thrombogenicity that is frequently observed in severe Covid-19. Based on our findings we expect that increasing MGP and protein S activity by vitamin K administration will considerably improve Covid-19 outcomes. An intervention trial to confirm this hypothesis is currently ongoing. Twelve patients have received treatment and there have been no unexpected safety issues. Prior to this trial, we already treated a cohort of hospitalized COVID-19 with vitamin K. ICU admission rate and mortality were significantly lower than in previous COVID-19 cohorts from our hospital.

The present invention therefore broadly relates to a new and completely unexpected application of vitamin K, wherein vitamin K preferably is administered to a mammalian subject for a prolonged period of time as a prophylactic or therapeutic agent to prevent, decrease and/or counteract Covid-19 disease or a similar disease.

The form of vitamin K used for preventing, decreasing and/or counteracting Covid-19 disease or a similar disease is either phylloquinone (vitamin K1) or menaquinone (vitamin K2). Compared to phylloquinone, menaquinones are more prone to improve the vascular vitamin K status. Thus, the active ingredient for use according to the invention is preferably selected from one of the menaquinones and combinations thereof, and most preferably it is selected from the long-chain menaquinones MK-7, MK-8, MK9 or MK-10. In certain embodiments of the invention, the vitamin K form used is MK-7. Also encompassed are any combinations of the long-chain menaquinones MK-7, MK-8, MK-9 or MK-10. Menaquinones are used in oral formulations, phylloquinone is used both orally and parenterally, in particular intravenously.

Vitamin K for use in the present invention will preferably be administered in addition to the normal dietary intake of vitamin K. Depending on the normal dietary intake of a given subject and the vitamin K status of this subject before treatment (i.e. at baseline), the dose of vitamin K to be administered according to the invention to achieve the desired effect of preventing, decreasing and/or counteracting Covid-19 disease or a similar disease, in other words the “effective amount” of vitamin K, will vary within certain limits. Typically, an effect will be seen when administering an amount of vitamin K, preferably menaquinone(s), in the range of between 5 and 10.000 µg/day, preferably between and 25 and 2.000 µg/day, more preferably between 50 and 1.000 µg/day, and most preferably between 100-500 µg/day.

According to some embodiments of the invention, vitamin K, in particular at least one menaquinone, may be prepared in the form of a concentrate. Typical examples of this approach are (i) the preparation of vitamin K by organic synthesis, followed by standard purification techniques including chromatography and crystallization and (ii) microbial production, e.g. deep tank fermentation, which is known in the art. These vitamin K products, in particular menaquinone products, have the advantage that they have a controlled constant quality, can be obtained at reasonable costs and can easily be incorporated in pharmaceutical or nutraceutical products without negatively affecting the taste.

Products resulting from organic synthesis may be used in a pure form, wherein “pure” means that the isolation product contains ≥ 80%, preferably ≥ 90%, ≥ 95%, ≥ 98% or ≥ 99.5 % by weight phylloquinone, menaquinone(s) or mixtures thereof and, consequently, ≤ 20% preferably ≤ 10%, ≤ 5%, ≤ 2%, or ≤ 0.5% by weight of other constituents.

Products resulting from microbial fermentation may be used in a pure form or a partially purified form, wherein partially purified means vitamin K concentrations ranging between 0.1% to 20% (w/w). The vitamin K concentrates may be used as such or added to a pharmaceutical or nutraceutical formulation, e.g. those described herein. Further, they may be used for fortifying food products.

Inflammation may be the result of both the genetic makeup and an acquired immune response, and is typically accompanied by increased production of cytokines, including TNF-alpha, interleukin-1 and interleukin-6, which are all related to the cytokine storm in COVID-19, as well as proteases, such as matrix metalloproteinases (MMPs) and neutrophil elastase. Hyperinflammation and subsequently proteolytic lung destruction and respiratory failure due to pneumonitis and acute respiratory distress syndrome (ARDS) is a major cause of death in COVID-19. Our finding of the strong correlation between dp-ucMGP and IL-6 levels underscores the role of vitamin K insufficiency in this pathological process.

Individuals with severe SARS-CoV-2 infections often have comorbidities that are also associated with reduced vitamin K status, such as hypertension, diabetes and cardiovascular diseases.^{10,20} We presumed that vitamin K deficiency would worsen Covid-19 outcome. The body uses vitamin K very efficiently, and storage capacity is low.²¹ There are reasons to suspect enhanced utilization of vitamin K for carboxylation of pulmonary MGP and coagulant factors in Covid-19.^{4,8,22-24} Depletion of vitamin K may have devastating consequences in lungs,²⁵ and it has been suggested that these effects may be very acute.²⁶

In a Czechs’ cohort of 2651 individuals, the dp-ucMGP levels were measured. Between 2008 and 2013, dp-ucMGP levels in patients were measured for research purposes in this study, 130 patients underwent PCR for COVID-19 in 2020 and were included in the study. 18 patients were positive for COVID-19, 112 patients PCR’s were tested negative. Of 18 patients with COVID-19, 10 patients were hospitalized and 8 patients were not. The mean of dp-ucMGP levels of hospitalized patients was not significantly different compared to not hospitalized COVID-19 positive patients (p=0.762).

We demonstrated that vitamin K deficiency develops during COVID-19 infection and is not only the result of poor vitamin K status prior to contracting SARS-CoV-2.

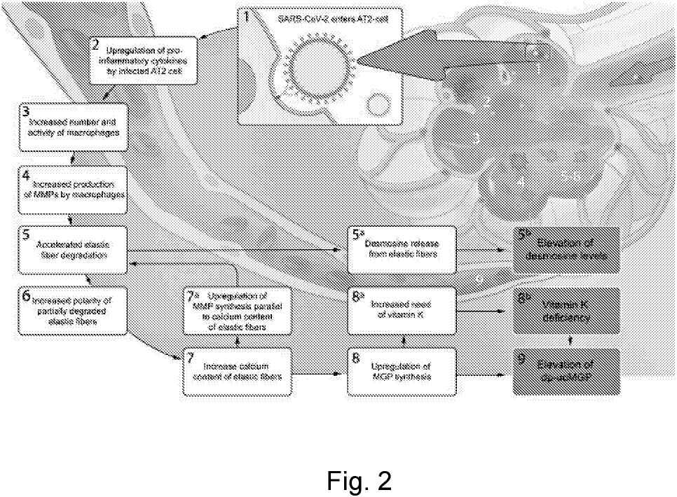

Preliminary data show that a subset of pulmonary macrophages, which produce MMPs and play a role in lung fibrosis,¹⁸ are increased in severe SARS-CoV-2 pneumonia.¹⁹ We started from the assumption that Covid-19 may be linked to both vitamin K deficiency and elastic fiber metabolism through a series of sequential pathologic steps, as illustrated in FIG. 2. We then evaluated whether a reduced vitamin K status would play a role in the pathogenesis of Covid-19 thereby linking, in particular, pulmonary and coagulopathic disease manifestations.

We demonstrated severely reduced extrahepatic vitamin K status in hospitalized Covid-19 patients. Impaired MGP activation was found to be associated with poor outcome and accelerated elastic fiber degradation. Procoagulant FII activity remained preserved in the majority of Covid-19 patients, which is compatible with the increased thrombogenicity that is frequently observed in severe Covid-19.

Low dietary vitamin K intake and VKA use are evident causes of vitamin K shortage.^{21,34} However, we have demonstrated that ongoing pathological processes leading to upregulation of vitamin K-dependent protein production and causing accelerated utilization of vitamin K for carboxylation is another important reason for severe vitamin K extrahepatic insufficiency in Covid-19.

Intriguingly, many comorbid conditions, which we and others found to be related to worse Covid-19 clinical outcomes, are associated with compromised vitamin K status.^{10,20,27} The same holds true for ageing.^{20,35} Vitamin K insufficiency is irrefutably linked to vascular calcifications by reducing active MGP levels required for inhibition of elastic fiber mineralization.^{9,10} Circumstantial evidence suggests that similar processes also occur in lungs.^{11,12,23-26} There seems to be an association between vascular mineralization and lung pathologies, as both lung fibrosis and emphysema are associated with arterial calcification scores.^{36,37} Calcification and degradation of elastic fibers are closely related pathological processes.¹⁵ This is illustrated by the strong correlation between circulating DES levels, which reflect the rate of systemic elastic fiber degradation, and arterial calcification score in COPD.³⁸ Furthermore, both elastic fiber degradation and arterial calcification are related to all-cause mortality in COPD patients underscoring the clinical relevance of these biomarkers.^{38,39} We demonstrated accelerated elastic fiber degradation in Covid-19 and a correlation of circulating dp-ucMGP with desmosine levels, suggesting an interrelationship between vitamin K shortage, insufficient MGP carboxylation and elastic fiber degradation in Covid-19 patients. We also found enhanced thoractic aortic calcification scores on CT in Covid-19 patients with poor prognosis, reflecting preexisting elastic fiber dysfunction. Vitamin K insufficiency could therefore represent a unifying risk factor for Covid-19 disease severity. Hypertension, diabetes, cardiovascular disease and older age are associated with remodeling of elastic tissues.¹⁰ These damaged and calcified elastic fibers are more prone to further degradation than intact fibers.^{15,40} We speculate that this pre-existing elastic fiber dysfunction renders them more susceptible to degradation following enhanced MMP production by macrophages during Covid-19.^{18,19}

We did not find a significant correlation between vitamin K status and pneumonia severity on CT. There are various possible explanations for this lack of association. Vitamin K insufficiency in Covid-19 patients is most likely the result of premorbid status and acute modifications secondary to the infection. It is plausible that SARS-CoV-2 infected patients with comorbid conditions develop respiratory failure with less lung involvement than those who are otherwise healthy. Furthermore, CT severity is a dynamic process that may change on a day-to-day basis.⁴¹ A clinical trial in which change of both vitamin K status and CT severity are simultaneously assessed before and after vitamin K supplementation would be a more suitable method to determine the effect of vitamin K on SARS-CoV-2 pneumonia.

Dp-ucMGP is associated to mortality in various cohorts.⁴² Vitamin K supplementation has a reducing effect on dp-ucMGP levels;^{34,43,44} the opposite holds true regarding VKA use.³⁴ Administration of vitamin K has previously demonstrated favorable effects on clinically relevant outcome measures.^{43,44} We found very high levels of dp-ucMGP in Covid-19 patients with poor prognosis. It may be expected that vitamin K administration has an improving effect on vitamin K status in Covid-19 patients, this, however, has never been studied. Additionally, it remains to be evaluated whether improving vitamin K status would result in a better prognosis in Covid-19 patients.

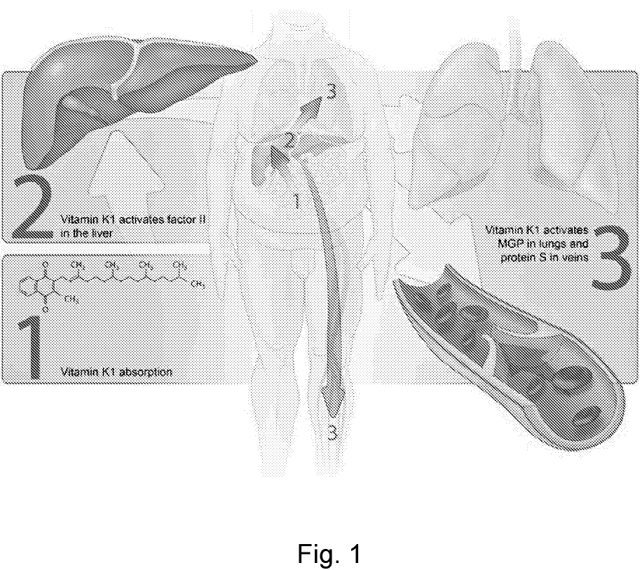

Vitamin K1, the main source of vitamin K in The Netherlands,⁴⁵ is preferentially transported to the liver, implying that the grade of carboxylation is usually higher for hepatic than extrahepatic vitamin K-dependent proteins (FIG. 1).^{6,7,46} This may be the reason that dp-ucMGP was severely elevated, while PIVKA-II was normal in the majority of Covid-19 patients. Furthermore, we assume that vitamin K insufficiency in Covid-19 patients has greater effects on protein S than on FII production (FIG. 1). This would be compatible with enhanced thrombogenicity in Covid-19.⁵ Preferred vitamin K-dependent activation of hepatic procoagulation factors over endothelial protein S would be compatible with findings from an autopsy series revealing bilateral deep venous leg thrombosis in all thromboembolic cases, as well as with thrombosis of the prostatic venous plexus in the majority of men who died of Covid-19.⁴⁷ Although increased thrombosis risk in a state of vitamin K insufficiency may sound paradoxical, this phenomenon has previously been described in calciphylaxis, a rare and life-threatening disorder.⁸ Calciphylaxis is characterized by the occlusion of cutaneous blood vessels due to calcification, leading to ischemic infarction of the skin.⁸ It is noteworthy that increased levels of inactive MGP are found in skin tissues and increased circulating levels of dp-ucMGP are noticed in calciphylaxis patients.^{8,48} Anticoagulant activity is impaired in calciphylaxis, similar to what we found in Covid-19, with thrombosis of microvessels as key histopathological features of both calciphylaxis and Covid-19 in skin and lungs, respectively.^{8,49}

VKA form a class of anticoagulant drugs that reduce the activity of procoagulation factors, as well as of other vitamin K-dependent proteins, by interfering with vitamin K metabolism. In line with our findings of compromised anticoagulant and relatively spared procoagulant activity during vitamin K insufficiency in Covid-19, stroke risk paradoxically increases in the first days following VKA initiation in atrial fibrillation patients.⁵⁰ Calciphylaxis risk and mortality is also significantly increased by VKA use,⁸ and VKA is related to reduced survival in idiopathic pulmonary fibrosis.²⁵ A proof-of-concept study on vitamin K1 supplementation in calciphylaxis is currently ongoing.⁸

The major strengths of our study are the thorough characterization of the Covid-19 patients included, use of robust biomarkers to quantify hepatic and extrahepatic vitamin K status, automated assessment of CT scans, and presentation of data suggesting relevant underlying disease mechanisms. However, there were also some limitations that should be addressed. It was impossible to determine which proportion of circulating dp-ucMGP and DES levels originated from the lungs, as both biomarkers are not tissue specific. There is urgent need for experimental data to better link vitamin K insufficiency specifically with Covid-19-related lung pathologies. Furthermore, we did not have the availability of a test to quantify protein S levels that have not been activated by vitamin K. Given the extreme extrahepatic vitamin K insufficiency in Covid-19, however, it seems reasonable to assume that carboxylated protein S levels in Covid-19 patients are reduced. As low vitamin K levels are found in comorbidities that are related to poor outcome of Covid-19,^{10,20} another limitation is that we were unable formally to determine whether vitamin K insufficiency truly predisposes patients to the development of severe Covid-19 or whether it is merely an epiphenomenon. However, the latter seems highly unlikely given the extreme elevation of dp-ucMGP levels in Covid-19 patients, which was much more pronounced than in hypertensive, diabetic and cardiovascular patients without Covid-19 (Supplementary Table 1). The strong correlation, which we found between vitamin K status and the rate of elastic fiber degradation, also suggests causality. We had to make use of a historical control group, due to the implementation of quarantines and social distancing practices to contain the Covid-19 pandemic. We do not consider this to be a major problem, however, as dp-ucMGP levels of our historical controls were poorer than previously reported in large groups of controls (Supplementary Table 2). Furthermore, differences in dp-ucMGP levels between Covid-19 patients and controls were of such a magnitude that loss of significance when comparing to a matched control group would be highly unlikely.

In conclusion, extrahepatic vitamin K status was severely compromised in Covid-19 and lower in patients with a poor outcome compared to those with good outcome. Covid-19 patients with premorbid elastic fiber pathologies appeared, in particular, to be at increased risk of complicated disease course. Extrahepatic/procoagulant prothrombin activation remained preserved. The data provided suggest potential mechanistic links between reduced vitamin K status, lung tissue injury and thrombogenicity in Covid-19. An intervention trial is now needed to assess whether vitamin K administration improves outcome in patients with Covid-19.

### Methods

### Subjects

134 subjects hospitalized for Covid-19 in the Canisius-Wilhelmina Hospital in Nijmegen, The Netherlands, between March 12^{th} and April 11^{th} 2020 were included for analysis. SARS-CoV-2 infection was confirmed by Real Time polymerase chain-reaction (RT-PCR) testing in all study subjects. Data on patient comorbidities were extracted from hospital admission records, and vitamin K antagonist (VKA) usage was determined based on records from pharmacies and anticoagulant clinics. The study was approved by the United Medical Research Ethics Committees of the Canisius-Wilhelmina Hospital (CWZ-nr. 027-2020; date of approval 12^{th} March 2020). The need for written informed consent was waived by the committee. There was, however, an opt-out possibility for patients after they were informed about the study.

A total of 184 age-matched control subjects from a previous COPD study were included in addition (www.controlled-trials.com, identifier ISRCTN86049077).²⁷ Covid-19 and control subjects where use of VKA was unknown were excluded from the analysis.

Patients were followed-up until they reached one of three endpoints: 1) discharge from the hospital, 2) admission to the intensive care unit (ICU) for intubation and mechanical ventilation, or 3) death. Outcome of Covid-19 patients was categorized as “good” if they were discharged from the hospital without the need for invasive ventilation, and “poor” if they either required intubation and mechanical ventilation or died due to Covid-19.

### Quantification of Dp-UcMGP

Although technically feasible, direct quantification of blood vitamin K levels would not have been an appropriate method to assess overall vitamin K status in our study due to differences in bioavailability and half-life time between the two naturally occurring vitamin K forms (i.e. vitamin K1 and K2). Additionally, the intake of vitamin K2, a group name of all menaquinones, is too low to measure accurately. Measuring inactive levels of vitamin K-dependent protein in the circulation is a valuable method for quantifying the combined deficit of vitamin K1 and K2. Desphospho-uncarboxylated (dp-uc)MGP (i.e. inactive MGP) may be considered as the most appropriate surrogate marker of extrahepatic vitamin K status in Covid-19.^{23,24} Subjects with high dp-ucMGP levels have low extrahepatic vitamin K status and vice versa.

Circulating dp-ucMGP levels were determined in EDTA plasma using the commercially available IVD CE marked chemiluminescent InaKif MGP assay on the IDS-iSYS system (IDS, Boldon, UK).²⁸ In brief, 50 µL of patient sample or calibrators were incubated with magnetic particles coated with murine monoclonal dpMGP antibody, an acridinium labelled murine monoclonal ucMGP antibody and assay buffer. The magnetic particles were captured using a magnet and a wash step performed to remove any unbound analyte. Trigger reagents were added. The resulting light emitted by the acridinium label is directly proportional to the concentration of dp-ucMGP in the sample. The within-run and total precision of this assay were 0.8 - 6.2% and 3.0 - 8.2%, respectively. The assay measuring range is between 200 - 12,000 pmol/L and was found to be linear up to 11,651 pmol/L.

Maximum dp-ucMGP’s were used for comparisons between groups, and baseline values were used for correlations of dp-ucMGP with blood and radiological biomarkers. Dp-ucMGP values below 300 pmol/L are considered to be in the normal healthy range.

### PIVKA-II

Protein induced by vitamin K absence (PIVKA)-II (i.e. ucFII) was used to assess hepatic/procoagulant vitamin K status. Subjects with high PIVKA-II levels have low hepatic vitamin K status and vice versa.

Circulating PIVKA-II levels were measured using a conformation-specific monoclonal antibody in an ELISA-based.²⁹ Results are expressed as arbitrary units per liter (AU/mL) as in states of vitamin K deficiency circulating ucFII may comprise multiple forms of partially carboxylated FII and neither their relative abundance in serum nor their relative affinity for the antibody is known. Using electrophoretic techniques 1 AU is equivalent to 1 mg of purified ucFII. The detection limit, as well as upper limit of normal, was 0.15 AU/mL ucFII in serum;²⁹ 0.15-0.5 AU/mL is mildly, 0.5-2.0 moderately and >2.0 is severely elevated.

### Desmosine

Plasma (p) desmosine and isodesmosine (DES) levels were used as a marker for the rate of elastic fiber degradation.³⁰ DES are formed during the cross-linking of tropo-elastin polymers and are released in the bloodstream after degradation of elastic fibers.^{30,31} pDES is therefore positively associated with the rate of systemic elastic fiber degradation.

DES fractions were measured using liquid chromatography-tandem mass spectrometry with deuterium-labelled desmosine as internal standard, as previously described.^{27,30} Coefficient of variations of intra- and inter-assay imprecision were <8.2%, lower limit of quantification of 140 ng/L, and assay linearity up to 210,000 ng/L.

For each pDES measurement in a Covid-19 patient, virtual age-matched pDES values were calculated using published pDES equations: (50+2.91*age for never-smokers and 70+3.12*age for ever smokers).³⁰

### CT Acquisition

Thin slice CT scans were acquired by using a Philips Ingenuity multi-detector row scanner (Philips Healthcare). CT images of 1-mm thickness were reconstructed by using iterative model-based reconstruction in the axial plane. A low-dose scanner protocol was used with 100 kVp and variable mAs without intravenous contrast administration.

### CT Lung Assessment

Quantitative measurements of the volume of ground glass and consolidation were undertaken using the Intellispace Portal (COPD package, Instellispace version 10, Philips Healthcare). In the software, first the lungs were segmented from the chest wall and major vessels and bronchi. Manual adjustments were implemented by a board-certified chest radiologist where required, given the extensive lung consolidation. Subsequently, the lung voxels were counted to derive a total lung volume in milliliters. Diseased lungs were defined as those voxels with an attenuation of Hounsfield Units (HU) > -700 as previously defined for interstitial lung disease.³² Visually this corresponded favorably to the COVID related abnormalities. The abnormal voxels were expressed as a percentage of the total volume as a percentage diseased lung. Additionally, a percentile method was employed, where the HU value at the 85th percentile was used.³³ Given that air has a HU of -1000 and water a HU of 0, the more the lung is diseased, the higher the HU value.

### CT Vascular Assessment

Coronary and aortic calcifications were quantified in the Intellispace Portal (Heartbeat CS package, Instellispace version 10, Philips Healthcare). Calcifications were defined as dense areas with a HU of 130 and higher. The calcifications were visually localized up to the arterial wall by a board-certified chest radiologist, who semi-automatically segmented the calcifications. The volume of calcifications was used as a measure of calcification burden.

### Statistical Analysis

Statistical analyses were performed using SPSS (version 24, IBM, Chicago, IL, USA). Analysis of variance (ANOVA) was used to compare dp-ucMGP levels between Covid-19 patients and controls as well as to compare dp-ucMGP and radiological scores between Covid-19 patients with good and poor outcomes, respectively. In subjects with Covid-19, the correlation between dp-ucMGP and pDES was assessed using Pearson’s correlation coefficient. Pearson’s correlation coefficient was also used for the association between dp-ucMGP and Covid-19 severity score, coronary artery calcium (CAC) score and thoracic aortic calcium (TAC) score on CT. Full factorial (including all interactions for fixed factors) analysis of covariance (ANCOVA) was used to perform aforementioned dp-ucMGP and radiological analyses adjusted for age, gender and use of VKA. pDES was adjusted for age in the comparison between Covid-19 patients and reference values as well as between Covid-19 patients with good and poor outcomes.

Dp-ucMGP, pDES and radiological scores had a log-normal distribution and were therefore natural log-transformed prior to analyses. The mean difference and 95% Cl of the log-transformed values was back-transformed to the mean fold change.

Use of VKA is associated with extremely high PIVKA-II and, therefore, users of VKA were separately assessed in the analysis with PIVKA-II as variable. Dialysis has a strong influence on pDES, and therefore, patients receiving dialysis at baseline were excluded from analysis involving pDES.

Normally distributed continuous variables are presented as mean ± standard deviation (SD), whereas continuous variables with a natural-log distribution were presented as back-transformed mean and 95% Cl. A P-value of <0.05 was used as threshold for statistical significance.

### Results

The mean age of COVID-19 patients was 68±12 years, 93 (70%) were male and 12 (9.0%) used VKA. Of the historical controls, 85 (46%) were male, 3 subjects (1.6%) were currently taking VKA, and mean age was 61±6.5 years. Characteristics are shown in Table 1 below.

TABLE 1

| | COVID-19 | | Controls |
|---|---|---|---|
| | Good outcome | Poor outcome | |
| Subjects | 64 | 59 | 184 |
| Age (years) | 64±13 | 72±9.8 | 61±6.5 |
| Male (%) | 41 (64) | 46 (77) | 85 (46) |
| VKA use (%) | 4 (6.3) | 7 (12) | 3 (1.6) |
| Hypertension (%) | 27 (42) | 22 (37) | 41 (22) |
| Diabetes mellitus (%) | 14 (22) | 14 (24) | 6 (3.3) |
| Cardiac or cardiovascular disease (%) | 16 (25) | 20 (34) | 10 (5.4) |
| Asthma/COPD (%) | 13 (20) | 12 (20) | 0 (0) |
| Other respiratory disease (%) | 5 (7.8) | 8 (14) | - |
| Immunocompromised (%) | 4 (6.3) | 2 (3.4) | - |
| Dialysis dependent (%)* | 1 (1.6) | 2 (3.4) | |
| Active malignancy (%) | 5 (7.8) | 6 (10) | 0 (0) |

Covid-19: Coronavirus 2019; VKA: Vitamin K antagonist; COPD: chronic obstructive pulmonary disease; * Systolic blood pressure >140 mmHg or diastolic blood pressure >90 mmHg;
** At admission

### Dp-ucMGP

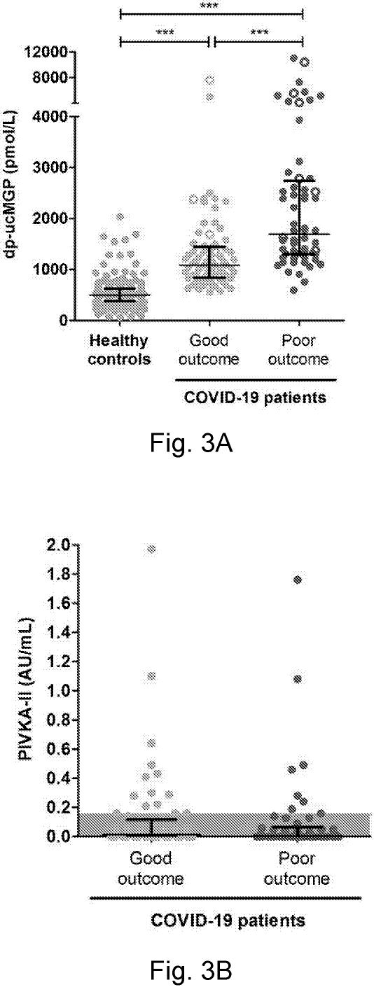

Dp-ucMGP levels were significantly higher in Covid-19 patients (1482 pmol/L, 95% Cl, 1346 to 1633 pmol/L) compared to healthy controls (471 pmol/L, 95% Cl, 434 to 511 pmol/L, mean fold change 3.15, 95% Cl, 2.78 to 3.58, P<0.001, FIG. 3A), which remained significant after adjustments (P=0.001). Dp-ucMGP levels were significantly higher in Covid-19 patients with poor outcome (1998 pmol/L, 95% Cl, 1737 to 2298 pmol/L) compared to those with good outcome (1163 pmol/L, 95% Cl, 1027 to 1319, mean fold change 1.72, 95% Cl, 1.42 to 2.07, P<0.001; FIG. 3A), and significance was maintained after adjustments (P=0.002).

### PIVKA-II

PIVKA-II levels were normal in 81.8%, mildly elevated in 14.0% and moderately elevated in 4.1% of Covid-19 patients not using VKA (FIG. 3B). In Covid-19 patients with good outcome and not using VKA, PIVKA-II levels were normal in 79.4%, mildly elevated in 16.2% and moderately elevated in 4.4%. In Covid-19 patients with poor outcomes and not using VKA, PIVKA-II levels were normal in 84.9%, mildly elevated in 11.3% and moderately elevated in 3.8%.

PIVKA-II levels were severely elevated in 100% of Covid-19 patients using VKA.

### Desmosine

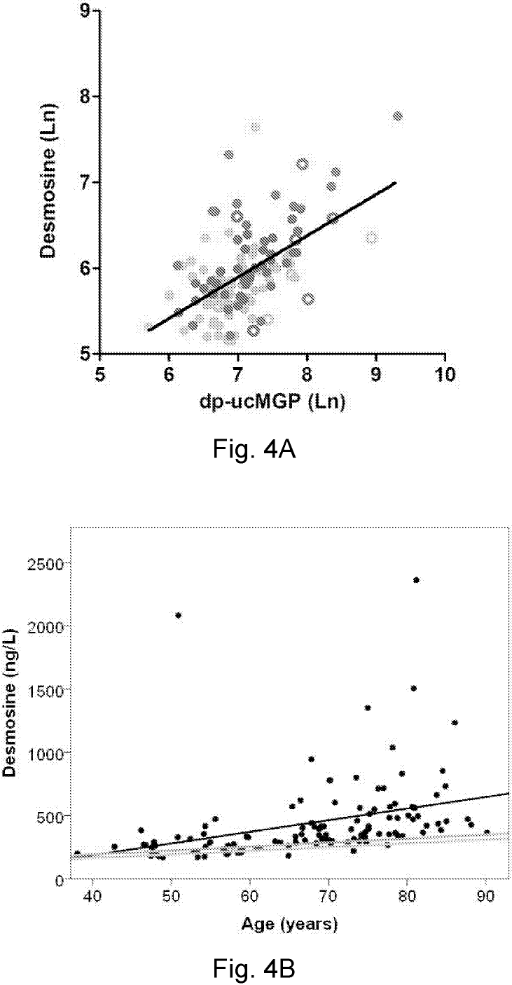

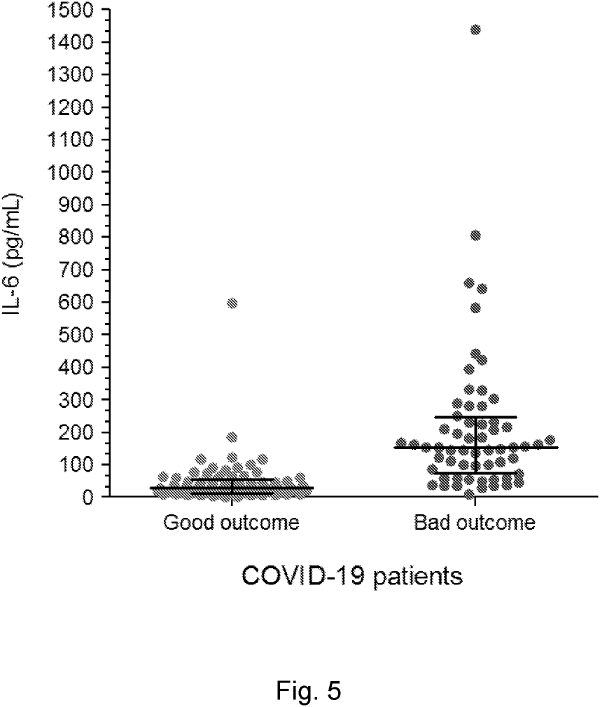

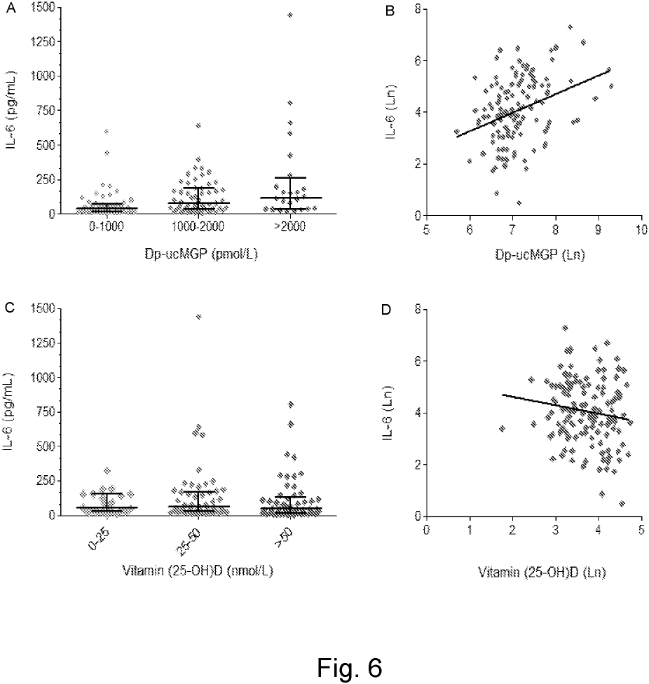

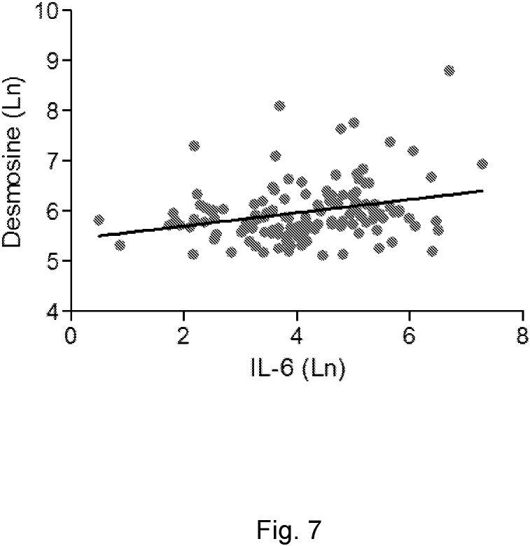

pDES levels were significantly higher in Covid-19 patients (0.38 ng/L, 95% Cl, 0.35 to 0.40 ng/L) compared to age-dependent reference values of never-smokers (0.24 ng/L, 95% Cl, 0.23 to 0.26 ng/L; mean fold change 1.55, 95% Cl, 1.41 to 1.71, P<0.001) and former or current smokers (0.28 ng/L, 95% Cl, 0.26 to 0.30 ng/L, mean fold change 1.36, 95% Cl 1.24 to 1.50, P<0.001; FIG. 4A).³⁰ pDES levels, corrected for age, were significantly higher in Covid-19 patients with poor (0.43 ng/L, 95% Cl 0.38 to 0.48 ng/L) compared to good outcomes (0.34 ng/L, 95% Cl 0.31 to 0.38 ng/L; mean fold change 1.26, 95% Cl, 1.08 to 1.48, P=0.004).

Dp-ucMGP was significantly associated with pDES (n=123, r=0.51, P<0.001; FIG. 4B).

### CT Assessment

Percentage pneumonia involvement was significantly higher in Covid-19 patients with poor (29.1%, 95% Cl, 24.9 to 34.2%) vs. good outcome (21.0%, 95% Cl, 18.2 to 24.2%, mean fold change 1.39, 95% Cl, 1.12 to 1.72, P=0.003). TAC score was significantly higher in Covid-19 patients with poor (2053, 95% Cl, 1120 to 3763) vs. good outcome (754, 95% Cl, 402 to 1415, mean fold change 2.72, 95% Cl, 1.14 to 6.51, P=0.025), which remained significant after adjustments (P=0.019). CAC score was not significantly different between Covid-19 patients with poor (449, 95% Cl, 230 to 878) and good outcomes (235, 95% Cl, 115 to 481, mean fold change 1.92, 95% Cl, 0.72 to 5.11, P=0.19, P=0.092 after adjustments).

The association between pulmonary involvement on CT and dp-ucMGP levels was not significant (n=108; r= 0.023; P= 0.81). Dp-ucMGP levels were significantly associated with TAC scores (n=106; r= 0.39; P<0.001) but not with CAC scores (n=106; r=0.093; P=0.35).

### References

1. Munster VJ, Koopmans M, van Doremalen N, van Riel D, de Wit E. A Novel Coronavirus Emerging in China - Key Questions for Impact Assessment. N Engl J Med 2020; 382:692-4.

2. Gandhi RT, Lynch JB, Del Rio C. Mild or Moderate Covid-19. N Engl J Med 2020 Apr 24 (Epub ahead of print).

3. Guan WJ, Ni ZY, Hu Y, et al. Clinical Characteristics of Coronavirus Disease 2019 in China. N Engl J Med 2020;382:1708-20.

4. Tang N, Li D, Wang X, Sun Z. Abnormal coagulation parameters are associated with poor prognosis in patients with novel coronavirus pneumonia. J Thromb Haemost 2020; 18:844-7.

5. Cui S, Chen S, Li X, Liu S, Wang F. Prevalence of venous thromboembolism in patients with severe novel coronavirus pneumonia. J Thromb Haemost 2020 Apr 9 (Epub ahead of print).

6. Fair DS, Marlar RA, Levin EG. Human endothelial cells synthesize protein S. Blood 1986;67:1168-71.

7. Booth SL, Martini L, Peterson JW, Saltzman E, Dallal GE, Wood RJ. Dietary phylloquinone depletion and repletion in older women. J Nutr 2003;133:2565-9.

8. Nigwekar SU, Thadhani R, Brandenburg VM. Calciphylaxis. N Engl J Med 2018; 378: 1704-14.

9. Luo G, Ducy P, McKee MD, et al. Spontaneous calcification of arteries and cartilage in mice lacking matrix GLA protein. Nature 1997;386:78-81.

10. Chatrou ML, Winckers K, Hackeng TM, Reutelingsperger CP, Schurgers LJ. Vascular calcification: the price to pay for anticoagulation therapy with vitamin K-antagonists. Blood Rev 2012;26:155-66.

11. Fraser JD, Price PA. Lung, heart, and kidney express high levels of mRNA for the vitamin K-dependent matrix Gla protein. Implications for the possible functions of matrix Gla protein and for the tissue distribution of the gamma-carboxylase. J Biol Chem 1988; 263:11033-6.

12. Price PA, Buckley JR, Williamson MK. The amino bisphosphonate ibandronate prevents vitamin D toxicity and inhibits vitamin D-induced calcification of arteries, cartilage, lungs and kidneys in rats. J Nutr 2001;131 :2910-5.

13. Abraham T, Hogg J. Extracellular matrix remodeling of lung alveolar walls in three dimensional space identified using second harmonic generation and multiphoton excitation fluorescence. J Struct Biol 2010;171:189-96

14. Rucker RB. Calcium binding to elastin. Adv Exp Med Biol 1974;48:185-209.

15. Basalyga DM, Simionescu DT, Xiong W, Baxter BT, Starcher BC, Vyavahare NR. Elastin degradation and calcification in an abdominal aorta injury model: role of matrixmetalloproteinases. Circulation 2004;110:3480-7.

16. Bouvet C, Moreau S, Blanchette J, de Blois D, Moreau P. Sequential activation of matrix metalloproteinase 9 and transforming growth factor beta in arterial elastocalcinosis. Arterioscler Thromb Vasc Biol 2008;28:856-62.

17. Lee JS, Basalyga DM, Simionescu A, Isenburg JC, Simionescu DT, Vyavahare NR. Elastin calcification in the rat subdermal model is accompanied by up-regulation of degradative and osteogenic cellular responses. Am J Pathol 2006;168:490-8.

18. Morse C, Tabib T, Sembrat J, et al. Proliferating SPP1/MERTK-expressing Macrophages in Idiopathic Pulmonary Fibrosis. Eur Respir J 2019;54:1802441.

19. Liao M, Liu Y, Yuan J, et al. The landscape of lung bronchoalveolar immune cells in COVID-19 revealed by single-cell RNA sequencing. Feb. 26, 2020 (https://www.medrxiv.org/content/10.1101/2020.02.23.20026690v1). preprint.

20. Zhou F, Yu T, Du R, et al. Clinical course and risk factors for mortality of adult inpatients with Covid-19 in Wuhan, China: a retrospective cohort study. Lancet 2020;395:1054-62.

21. Usui Y, Tanimura H, Nishimura N, Kobayashi N, Okanoue T, Ozawa K. Vitamin K concentrations in the plasma and liver of surgical patients. Am J Clin Nutr 1990;51 :846-52.

22. Alperin JB. Coagulopathy caused by vitamin K deficiency in critically ill, hospitalized patients. JAMA 1987;258:1916-9.

23. Booth AJ, Hadley R, Cornett AM, et al. Acellular normal and fibrotic human lung matrices as a culture system for in vitro investigation. Am J Respir Crit Care Med 2012;186:866-76.

24. Hardie WD, Korfhagen TR, Sartor MA, et al. Genomic profile of matrix and vasculature remodeling in TGF-alpha induced pulmonary fibrosis. Am J Respir Cell Mol Biol 2007;37:309-21.

25. Noth I, Anstrom KJ, Calvert SB, et al. A placebo-controlled randomized trial of warfarin in idiopathic pulmonary fibrosis. Am J Respir Crit Care Med 2012;186:88-95.

26. Alagha K, Secq V, Pahus L, et al. We should prohibit warfarin in idiopathic pulmonary fibrosis. Am J Respir Crit Care Med 2015;191:958-60.

27. Piscaer I, van den Ouweland JMW, Vermeersch K, et al. Low Vitamin K Status Is Associated with Increased Elastin Degradation in Chronic Obstructive Pulmonary Disease. J Clin Med 2019;8:1116.

28. Jaminon AMG, Dai L, Qureshi AR, et al. Matrix Gla protein is an independent predictor of both intimal and medial vascular calcification in chronic kidney disease. Sci Rep 2020;10:6586.

29. Belle M, Brebant R, Guinet R, Leclercq M. Production of a new monoclonal antibodyspecific to human des-gamma-carboxyprothrombin in the presence of calcium ions. Application to the development of a sensitive ELISA-test. J Immunoassay 1995;16:213-29.

30. Huang JT, Bolton CE, Miller BE, et al. Age-dependent elastin degradation is enhanced in chronic obstructive pulmonary disease. Eur Respir J 2016;48:1215-8.

31. Partridge SM, Elsden DF, Thomas J, Dorfman A, Telser A, Ho PL. Incorporation of labelled lysine into the desmosine cross-bridges in elastin. Nature 1966;209:399-400.

32. Salaffi F, Carotti M, Di Donato E, Di Carlo M, Ceccarelli L, Giuseppetti G. Computer-Aided Tomographic Analysis of Interstitial Lung Disease (ILD) in Patients with Systemic Sclerosis (SSc). Correlation with Pulmonary Physiologic Tests and Patient-Centred Measures of Perceived Dyspnea and Functional Disability. PLoS One 2016; 11: e0149240.

33. Ninaber MK, Stolk J, Smit J, et al. Lung structure and function relation in systemic sclerosis: application of lung densitometry. Eur J Radiol 2015;84:975-9.

34. Cranenburg EC, Koos R, Schurgers LJ, et al. Characterisation and potential diagnostic value of circulating matrix Gla protein (MGP) species. Thromb Haemost 2010;104:811-22.

35. Theuwissen E, Magdeleyns EJ, Braam LA, et al. Vitamin K status in healthy volunteers. Food Funct 2014;5:229-34.

36. Nathan SD, Weir N, Shlobin OA, et al. The value of computed tomography scanning for the detection of coronary artery disease in patients with idiopathic pulmonary fibrosis. Respirology 2011;16:481-6.

37. Bhatt SP, Nath HP, Kim YI, et al. Centrilobular emphysema and coronary arterycalcification: mediation analysis in the SPIROMICS cohort. Respir Res 2018;19:257.

38. Rabinovich RA, Miller BE, Wrobel K, et al. Circulating Desmosine Levels Do Not Predict Emphysema Progression but Are Associated With Cardiovascular Risk and Mortality in COPD. Eur Respir J 2016;47:1365-73.

39. Williams MC, Murchison JT, Edwards LD, et al. Coronary Artery Calcification Is Increased in Patients With COPD and Associated With Increased Morbidity and Mortality. Thorax 2014;69:718-23.

40. Umeda H, Aikawa M, Libby P. Liberation of desmosine and isodesmosine as amino acids from insoluble elastin by elastolytic proteases. Biochem Biophys Res Commun 2011;411:281-6.

41. Wang Y, Dong C, Hu Y, et al. Temporal Changes of CT Findings in 90 Patients withCOVID-19 Pneumonia: A Longitudinal Study. Radiology 2020 Mar 19 (Epub ahead of print).

42. Roumeliotis S, Dounousi E, Eleftheriadis T, Liakopoulos V. Association of the Inactive Circulating Matrix Gla Protein with Vitamin K Intake, Calcification, Mortality, and Cardiovascular Disease: A Review. Int J Mol Sci 2019;20:628.

43. Brandenburg VM, Reinartz S, Kaesler N, et al. Slower Progress of Aortic Valve Calcification With Vitamin K Supplementation: Results From a Prospective Interventional Proof-of-Concept Study. Circulation 2017;135:2081-3.

44. Knapen MH, Braam LA, Drummen NE, Bekers O, Hoeks AP, Vermeer C. Menaquinone-7 supplementation improves arterial stiffness in healthy postmenopausal women. A double-blind randomised clinical trial. Thromb Haemost 2015;113:1135-44.

45. Schurgers LJ, Vermeer C. Determination of phylloquinone and menaquinones in food. Effect of food matrix on circulating vitamin K concentrations. Haemostasis 2000;30:298-307.

46. Fair DS, Revak DJ. Quantitation of human protein S in the plasma of normal and warfarin-treated individuals by radioimmunoassay. Thromb Res 1984;36:527-35.

47. Wichmann D, Sperhake JP, Lütgehetmann M, et al. Autopsy Findings and Venous Thromboembolism in Patients With COVID-19: A Prospective Cohort Study. Ann Intern Med 2020 May 6 (Epub ahead of print).

48. Kramann R, Brandenburg VM, Schurgers LJ, et al. Novel Insights Into Osteogenesisand Matrix Remodelling Associated With Calcific Uraemic Arteriolopathy. Nephrol Dial Transplant 2013;28:856-68.

49. Ackermann M, Verleden SE, Kuehnel M, et al. Pulmonary Vascular Endothelialitis, Thrombosis, and Angiogenesis in Covid-19. N Engl J Med 2020 May 21 (Epub ahead of print).

50. Azoulay L, Dell’Aniello S, Simon TA, Renoux C, Suissa S. Initiation of Warfarin in Patients With Atrial Fibrillation: Early Effects on Ischaemic Strokes. Eur Heart J 2014;35:1881-7.

51. Mancia G, Rea F, Ludergnani M, Apolone G, Corrao G. Renin-Angiotensin-Aldosterone System Blockers and the Risk of Covid-19. N Engl J Med 2020 May 1 (Epub ahead of print).

1. A composition for use in preventing or counteracting COVID-19 disease and/or alleviating severe symptoms of said disease, said composition comprising a therapeutically active amount of vitamin K, either alone or in combination with one or more other therapeutically active agents,
wherein the use comprises administering said composition to a mammalian subject, either
1. as prophylactic agent in preventing or reducing the risk of developing a serious disease or mortality by COVID-19 in the said subject, or
2. as therapeutic agent in preventing that the said disease becomes more severe, or
3. as therapeutic agent in reducing the severity of the said disease.

2. A composition for use according to claim 1, wherein the composition for prophylactic use is a pharmaceutical formulation or a nutritional product and the composition for therapeutic use is a pharmaceutical formulation.

3. A composition for use according to claim 2, wherein the nutritional product is a beverage or a dietary supplement.

4. A composition for use according to claim 1, wherein the mammalian subject is a human.

5. A composition for use according to claim 1, wherein vitamin K is administered to the mammalian subject as a supplement to the normal daily intake of vitamin K containing food.

6. A composition for use according to claim 1, wherein the composition for prophylactic use is administered orally and the composition for therapeutic use is administered orally or parenterally.

7. A composition for use according to claim 1, wherein said vitamin K comprises phylloquinone (vitamin K1) or menaquinone (vitamin K2) or a combination thereof.

8. A composition for use according to claim 7, wherein menaquinone (“MK”) is selected from the group of MK-4, MK-7, MK-8, MK-9, MK-10, or a combination thereof.

9. A composition for use according to claim 1, wherein the composition is combined with a therapeutically active amount of vitamin D, in particular vitamin D3 (cholecalciferol).

10. A composition for use according to claim 1, wherein vitamin K is comprised for administration in the following daily dosage:
(a) when vitamin K is phylloquinone, 5-5.000 microgram (µg), preferably 50-4.000 µg, more preferably 200-2.000 µg and most preferably 400-1.000 µg;
(b) when vitamin K is menaquinone-4 (MK-4), 5-3.000 µg, preferably 50-2.000 µg, more preferably 100-1.000 µg and most preferably 200-500 µg;
(c) when vitamin K is any one of the long chain menaquinones (MK-7, MK-8, MK-9, or MK-10) is, 5-2.000 µg, preferably 25-1.000 µg, more preferably 50-1.000 µg and most preferably 100-500 µg.

11. A composition for use according to claim 1, wherein the daily dosage of vitamin D amounts to 500-1.000 IE, preferably 700-900 IE and more preferably about 800 IE.

12. A composition for use according to claim 1, wherein the composition for therapeutic use comprises vitamin K1 in an amount as defined in claim 10 (a), and the daily dosage is preceded by a bolus of vitamin K1 of 2-50 mg.

13. A composition for use according to claim 1, wherein the one or more other therapeutically active agents comprise antiviral, antimicrobial or anti-inflammatory agents or combinations thereof.

14. A composition for use according to claim 1, wherein the composition is administered to a subject during a COVID-19 pandemia or epidemia, wherein the administration to the subject is prolonged for at least one to 6 months after the disease, and preferably lifelong.

15. Diagnostic assay for estimating the risk of developing serious illness or mortality by COVID-19 disease in an individual, wherein the diagnostic assay comprises:
(a) assessing the vitamin K status in blood, plasma or serum of the individual, who is infected or potentially infected with SARS-CoV-2 or a similar microbe,
(b) providing a reference scheme showing the vitamin K status of a reference population of healthy people, wherein the vitamin K status was determined in the same way as in step a),
(c) comparing the value obtained in step (a) with the reference scheme of step (b), wherein a value outside the normal range of the reference scheme is indicative for a higher risk of developing a serious disease or mortality by COVID-19 or a similar infectious disease.

16. Diagnostic assay according to claim 15, wherein the vitamin K status is determined by measuring the degree of carboxylation of circulating extrahepatic Gla-proteins in blood, plasma, serum or another body fluid of a subject, in particular dephospho-uncarboxylated matrix GLA-protein (“dp-ucMGP”) and uncarboxylated osteocalcin (“ucOC”).

17. Diagnostic assay according to claim 15, wherein the vitamin K status is determined in blood, plasma, serum or another body fluid using one or more of the following techniques:
(a) measuring the amount of despospho-uncarboxylated matrix Gla protein (dp-ucMGP) using a known technique, for example IDS-iSYS InaKtif or ELISA,
(b) measuring the amount of Proteins Induced by Vitamin K Absence (PIVKA),
(c) measuring the amount of phylloquinones and menaquinones,
(d) determining the ratio of uncarboxylated to carboxylated osteocalcin.

18. Diagnostic assay according to claim 15, wherein the assay further comprises:
(d) administering a composition comprising a therapeutically effective amount of vitamin K according to claim 15.

19-21. (canceled)

22. A method of preventing or treating COVID-19 disease in a patient in need thereof, the method comprising administering to the patient a composition comprising a therapeutically active amount of vitamin K to prevent or treat COVID-19 disease.

23. The method of claim 22, wherein the vitamin K comprises (a) phylloquinone in an amount of 5-5.000 micrograms (pg), 50-4.000 pg, 200-2.000 pg, or 400-1.000 pg; (b) menaquinone-4 (MK-4) in an amount of 5-3.000 pg, 50-2.000 pg, 100-1.000 pg, or 200-500 pg; or (c) any one of long chain menaquinones (MK-7, MK-8, MK-9 or MK-10) in an amount of 5-2.000 pg, 25-1.000 pg, 50-1.000 pg, or 100-500 pg.
